(19) United States
(12) Patent Application Publication
(10) Pub. No.: US 2017/0079625 A1
(43) Pub. Date: Mar. 23, 2017

(54) MOTION GATED-ULTRASOUND THERMOMETRY USING ADAPTIVE FRAME SELECTION

(71) Applicant: KONINKLIJKE PHILIPS N.V., EINDHOVEN (NL)

(72) Inventors: Shougang Wang, Ossining, NY (US); Ajay Anand, Fishkill, NY (US); Sheng-Wen Huang, Ossining, NY (US); Shriram Sethuraman, Woburn, MA (US)

(21) Appl. No.: 15/311,964

(22) PCT Filed: Apr. 3, 2015
(86) PCT No.: PCT/IB2015/052461

§ 371 Date: Nov. 17, 2016

Related U.S. Application Data
(60) Provisional application No. 62/002,239, filed on May 23, 2014.

(51) Int. Cl. A61B 8/08; A61N 1/06; A61B 8/00

(52) U.S. Cl. CPC A61B 8/5276; A61B 8/085; A61B 8/4209; A61B 8/5246; A61B 8/543; A61N 1/06

(57) ABSTRACT

Movement (204) of an object is detected and, based on the detected movement, imaging of the object is selectively commenced (228). The imaging is interrupted such that the commencing and interrupting result in temporally spaced apart (216) periods of the imaging. Content of images acquired in respectively different periods is compared (238), to match the images based on content. The movement may have a cyclical component. The object may include body tissue for ablating by applying energy from an energy source. The images to be compared can depict respective regions of the ablating, with the comparing being confined to outside the regions. The detecting, the selecting, the comparing, and the matching may be performable in real time. In one embodiment, an image has portions having respective spatial locations, and respective temperature values at the locations of the object are determined in forming a temperature map of the image. A temporal series of the maps, and optionally ultrasound B-mode images, are displayable in real time.

### FIELD OF THE INVENTION

The present invention relates to acquiring images during temporally spaced apart periods and, more particularly, to matching of the images.

### BACKGROUND OF THE INVENTION

Liver cancers are malignant tumors that grow on the surface of or inside the liver. Liver tumors are discovered with medical imaging equipment or present themselves symptomatically as an abdominal mass, abdominal pain, jaundice, nausea or liver dysfunction. There are a million new cases worldwide each year of primary liver cancer, 83% of which arise in developing countries. About half a million of the new cases are metastatic cancer, occurring mostly in the western hemisphere.

Recently, it has become possible to accurately target tumors anywhere in the body.

At present, the only reasonable chance to cure liver cancer is surgery, either with resection (i.e., removal of the tumor) or a liver transplant. If all known cancer in the liver is successfully removed, the patient will have the best outlook for survival. Surgery to remove part of the liver is called partial hepatectomy. It is feasible, if the person is healthy enough and all of the tumor can be removed while leaving enough healthy liver behind.

An alternative in widespread use, as a way of avoiding surgery, is radiofrequency ablation (RFA) for thermal treatment of tumors.

Current clinical applications manage to deliver a lethal dose of heat by means of an inserted heating electrode. The electrode can be introduced at the distal end of a radiofrequency needle. Body tissue is heated locally up to above 60° centigrade (C.), coagulating and thereby destroying the cancerous region.

During the procedure, the change in temperature is closely monitored to ensure treatment quality. The monitoring is preferably non-invasive.

Several temperature-monitoring techniques have been used historically.

Among these are the use of thermocouples mounted on the end of the radiofrequency needle and spatial monitoring with magnetic resonance imaging (MRI).

An advanced electrode consists of multiple tips, each of which can be separately controlled regarding its heat deposition.

Each tip has a thermocouple (i.e., tiny thermometer) incorporated, which allows continuous monitoring of tissue temperatures, and each tip's power is automatically adjusted so that the target temperatures remain constant.

An indication of the actually ablated tissue area is obtained for guidance in overriding the automatic adjustment. Power levels are thereby lowered in correspondence with achievement of objectives as to the extent of the ablation.

Ideally, the indication would effectively spatially distinguish the tissue that has already been ablated from the currently healthy or unablated tissue.

Commonly-assigned U.S. Pat. No. 8,328,721 to Savery et al. (hereinafter “Savery”) describes derivation of optical absorption coefficients for determining body function and structure.

In Savery, calculating the coefficients employs a temperature mapping module for forming temperature maps based on ultrasound interrogation.

For this purpose, acquisitions over time are compared and are preferably made with the same ultrasound imaging parameters. The description of the temperature mapping module, and the analysis underlying its functioning, in Savery are incorporated herein by reference.

When comparing imaging acquisitions over time, it is known to compensate for cyclical motion in the object being imaged.

### SUMMARY OF THE INVENTION

What is proposed herein below addresses one or more of the above concerns.

The distinguishing of ablated, from unablated, tissue would optimally be achieved through real-time monitoring of the in-vivo three-dimensional (3D) temperature distribution in the body.

Real-time monitoring of the in vivo 3D temperature distribution in the body can currently only be achieved with reasonable accuracy through magnetic resonance imaging (MRI).

However, using an MRI scanner as a 3D thermometer is very expensive.

Computed tomography (CT) can be used for the purpose of temperature measurement, but this is only possible to make a relatively coarse measurement of temperature change, i.e., one that is accurate only within 5° C.

For practical clinical applications, these methods have been limited by the limited spatial sampling (thermocouples), by the limited accuracy (CT), or by cost of the procedure (MRI).

Also, both for the above-described RF ablation, and for high-intensity focused ultrasound (HIFU) based ablation, motion of the body tissue in the region of interest limits the treatment precision and quality.

With regard to motion compensation, computed tomography CT, MM, and other motion gating systems use a fixed-time-delay trigger at a certain phase of the breathing cycle to compensate for body movement caused by respiration. The delay may be set to pick a particular phase cycle-to-cycle, to stabilize a monitored image based on imaging acquisition at a single phase.

However, such systems do not afford enough accuracy in ultrasound RF tracking based thermometry.

In particular, breathing motion is not consistent cycle to cycle, and more often is irregular.

It would be desirable for ultrasound data to be acquired instantly responsive to a fixed-time-delay trigger that is set off upon detection of a breathing cycle landmark, such as the peak value of each cycle.

However, if one were to use the signal level (e.g., each cycle peak) as a trigger, inherent delay would exist between detecting the level and triggering the ultrasound system; and the temperature calculation using the RF data received via ultrasound depends on precisely maintaining the position of the probe relative to the human body, breathing cycle to breathing cycle. Specifically, for effective temperature estimation, i.e., for an accurate temperature map based on successive images, the local temperature-induced strain, which is essentially a spatial gradient of apparent displacement, must be less than 0.5%.

Thus, in the hypothetical case of using the signal level as a trigger in ultrasound RF tracking based thermometry, the above-described inherent delay would cause, in view of the cycle-to-cycle irregularity likewise mentioned herein above, enough spatial movement to decorrelate, and thus degrade, the temperature maps.

Real-time monitoring of 3D temperature distribution, via a temporal series of temperature maps, would therefore be compromised.

This in turn would compromise the ablation monitoring.

What is proposed herein is directed to alleviating such compromise.

In accordance with what is proposed herein, movement of an object is detected. Based on the detected movement, imaging of the object is selectively commenced. The imaging is interrupted such that the commencing and interrupting result in temporally spaced apart periods of the imaging. Content of images acquired in respectively different periods is compared, to match the images based on content.

In a sub-aspect, the object includes body tissue for ablating by applying energy from an energy source.

In a further sub-aspect, the images to be compared depict respective regions of the ablating. The comparing is confined to outside the regions.

As a related sub-aspect, the detecting, the selecting, the comparing, and the matching are performed in real time. In this disclosure, “real time” means without intentional delay, given the processing limitations of the system and the time required to accurately perform the function.

In another aspect, a representation of the object's temperature distribution is updated in real time, and is displayable as a temporal series of temperature maps for monitoring the ablation.

Details of the novel, image matching between periodic, imaging-object-motion driven acquisitions are set forth further below, with the aid of the following drawings, which are not drawn to scale.

### BRIEF DESCRIPTION OF THE DRAWINGS

FIG. 1 is a schematic diagram of an exemplary image matching apparatus, in accordance with the present invention;

FIGS. 2 and 3 are conceptual diagrams providing examples of formulas and concepts relating to operation of the apparatus of FIG. 1; and

FIG. 4 is a set of flow charts demonstrating a possible operation for the apparatus of FIG. 1.

### DETAILED DESCRIPTION OF EMBODIMENTS

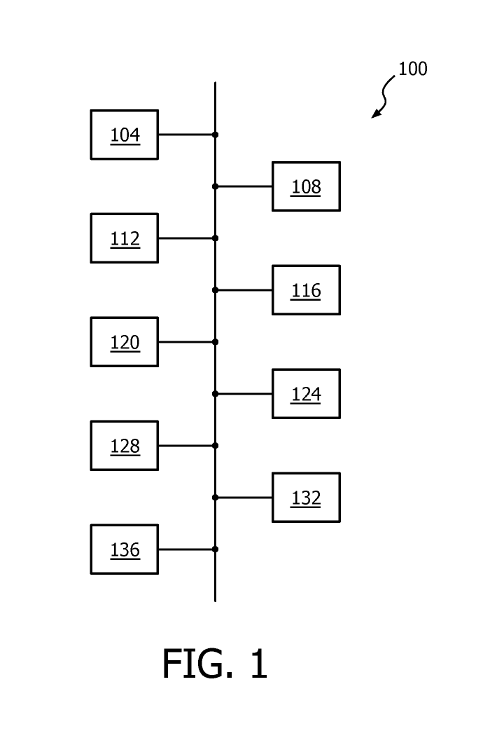

FIG. 1 depicts, by way of illustrative and non-limitative example, an image matching apparatus 100 usable for image-based matching between periodic, imaging-object-motion driven acquisitions and particularly in motion-gated ultrasound thermometry. The apparatus 100 includes a movement detection processor 104, image acquisition circuitry 108 such as that of an ultrasound scanner, an image matching processor 112, an image monitoring processor 116 such as that of an ultrasound scanner, an energy source 120 for applying energy to heat body tissue, a respiratory-phase sensor 124, and a respiratory belt 128 communicatively, e.g., physically, connected to the sensor. Further included are a respiration recording device 132 and an imaging probe 136.

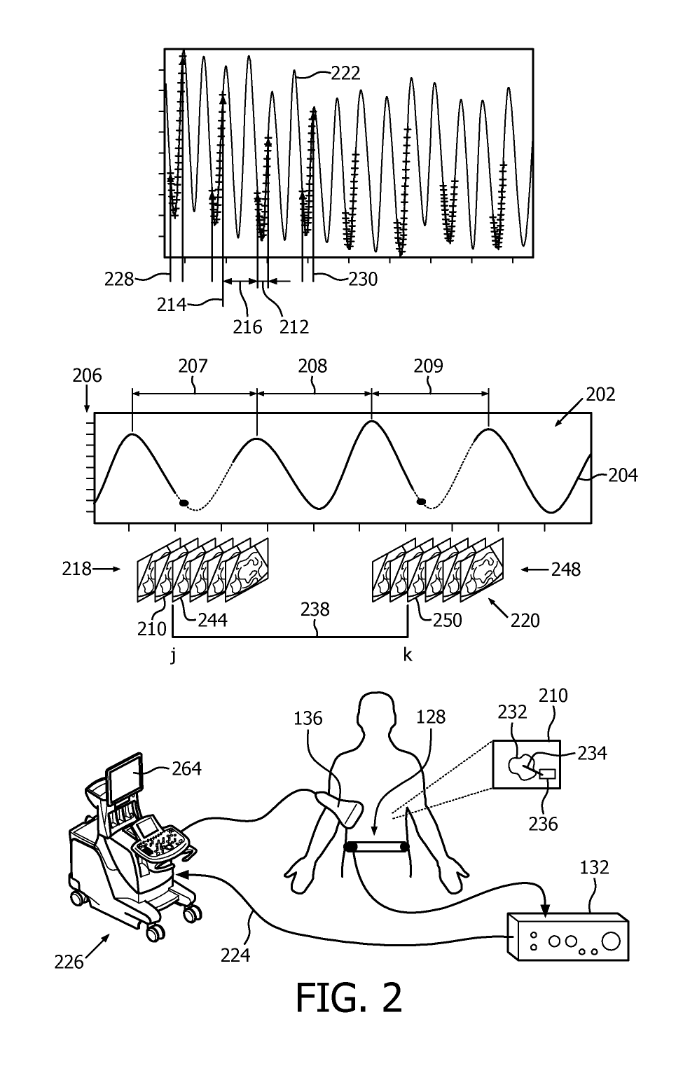

As seen in FIG. 2, movement of an object, such as the liver or a portion thereof, has a respiratory cyclical component 202 arising due to corresponding motion of the nearest lung. In a breathing plot, or “waveform”, 204, the range of the object's displacement 206 which is the ordinate varies over a cycle 207 along the abscissa. Subsequent cycles 208, 209 are also shown. The consecutive “+” signs in the plot 204 represent a sequence of frames 210. Each sequence constitutes a period 212 of acquisition. Each period 212 is terminated by an interruption 214 in the acquisition, resulting in spaced apart 216 periods. The acquired frames 210 of an acquisition period 212 will be referred to herein as a file 218. Another, subsequent file 220 is also shown. Each acquisition in FIG. 2 is preceded by a breathing cycle landmark, such as a local peak 222. The movement detection processor 104, which may be integrated with the respiration recording device 132 as a unit, includes a hardware or software subsytem for periodically, i.e., upon detecting a local peak 222, issuing a frame acquisition trigger 224 to the ultrasound scanner 226, to commence imaging acquisition. The issuance of the trigger 224 may occur a fixed time after detection of the peak 222 in the respiration cycle 208. The image acquisition circuitry 108 of the scanner 226 commences image acquisition, as shown by the commencement up arrow 228, and, a fixed time period later, interrupts 214 acquisition, thereby terminating the period 212, as shown by the interruption up arrow 230. Acquisition may occur for each cycle 207-209 or, as in FIG. 2, just for some cycles 207, 209, where the dot on the waveform 204 marks the start of acquisition for the current cycle. The negative peak of the valley just beyond each dot corresponds to a predefined phase. The periods of acquisition for the cycles 207-209 phase-wise overlap to thereby commonly contain phases, such as that predefined phase.

The respiratory belt 128 and a physically connected respiratory-phase sensor 124 are implementable as the belt 110/310 and the stretch transducer, respectively, of U.S. Patent Publication No. 2008/0109047 to Pless. The respiratory recording device 132 may be provided with the storage device 440 of Pless for storing the respiratory waveform 204 as it is acquired. The disclosure in Pless in paragraphs [0090]-[0014] is incorporated herein by reference. The respiratory recording device 132 detects the local peak 222 based on the constantly updated stored waveform 204.

As seen in FIG. 2, each acquired frame 210 depicts a region of ablation 232 formed by application of heat from the energy source 120, such as one or more ablation electrodes 234.

The frame-to-frame comparisons proposed herein are made, and confined to, outside the regions of ablation 232. An example of a region for comparison 236 which is outside the region of ablation 232 is shown for the acquired frame 210. Since the electrode(s) 234, and the surrounding region of ablation 232, tend to be centered in the frames 210, the region for comparison 236 can be preset as a fixed area of each frame sufficiently offset from center, e.g., near the periphery of the frame. The operator may define the region of comparison 236. This may be done interactively, for example, on screen.

An example of a frame-to-frame comparison 238, in the methodology proposed herein, is shown in FIG. 2 with respect to the two consecutive files 218, 220, although comparisons may be made between non-consecutive files. If, for example, N acquisition periods 212 each result in M frames 210 acquired, the frame-to-frame comparison 238 refers generally to a comparison between one frame j 244 of the first file 218 of a pair 248 of files and another frame k 250 of the second file 220 of the pair of files, with 1<j<M, and 1<k<M. The first file 218 of the pair 248 can, but need not necessarily, be the first file in the scan spanning the N files.

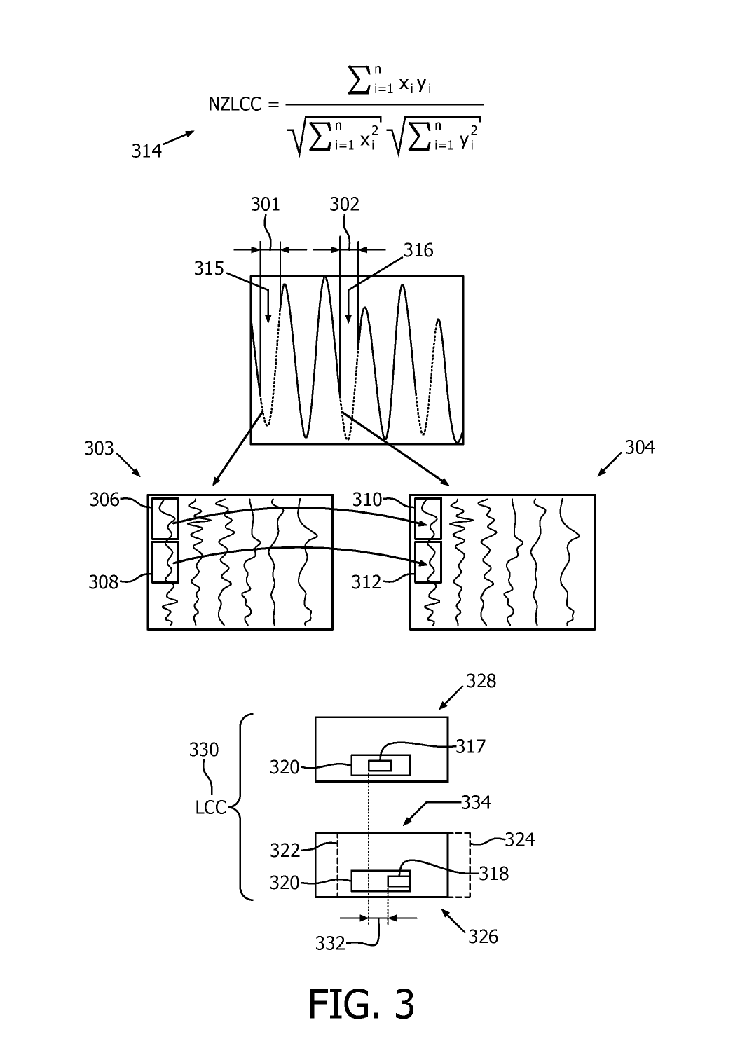

In a sample embodiment illustrated in FIG. 3, the comparison 238 is done piece-wise and is based on speckle matching. From different acquisition time periods 301, 302, two respective frames to be compared 303, 304 are chosen. Each frame 303, 304 may be divided, pixel-wise, into respective segments 306, 308, 310, 312. The segment 306 can have a width of one or more pixels. The frame 303 may have a length that accommodates, for instance, 8 or 9 segments. Each segment 306 in the first frame 303 is cross-correlated with its counterpart segment 310 in the second frame 304. A normalized zero-lag cross-correlation (NZLCC) 314 is employed.

$$NZLCC = \frac{{\sum }_{i = 1}^{n} {x}_{i} {y}_{i}}{\sqrt{{\sum }_{i = 1}^{n} {x}_{i}^{2}} \sqrt{{\sum }_{i = 1}^{n} {y}_{i}^{2}}}$$

The value xᵢ in the formula 314 is the brightness value of a pixel in one frame 303 and y₁ is the brightness value of the counterpart pixel in the other frame 304. The set of possible brightness values has been filtered to a range that is centered on zero. The summation in the formula 314 is done over the whole segment 306.

The correlation coefficient of the NZLCC 314 serves as a similarity index. It is in the range from −1 to 1. Value 1 represents that the two vectors {xᵢ}, {yᵢ} are identical. Value −1 represents that the two vectors are exactly opposite.

The similarity indices over all segments of the frame pair 303, 304 are averaged to arrive at a whole frame-pair similarity index.

The entries in an M×M matrix are filled with the M×M whole frame-pair similarity indices for the M frames 210 in each of the first two files 315, 316.

The two frames 210 corresponding to the highest-valued entry are deemed to be the best match between the two files 315, 316.

Both frames 315, 316 are selected as input for temperature map formation.

In some embodiments, no further frame selection is needed from the first file 315.

In one such embodiment, the selected frame 210 from the first file 315 serves as a reference frame for any subsequent speckle-based comparisons. In particular, the above procedure is repeated for the next file, i.e., third file, serving as the second file of the pair; however, only one frame of the first file 315 is considered, i.e, the reference frame already determined as described above. Accordingly, instead of an M×M matrix, a 1+M matrix, of whole frame-pair similarity indices is formed. The highest-valued entry determines the frame selection for the current file, i.e., third file. This same procedure, based on a 1+M matrix, is repeated for selecting a frame from the fourth file, serving as the second file of the pair; and the procedure is repeated each time for then current file, up to the Nth file. Accordingly, N frames 210 are selected in total. Temperature maps are formable between the reference frame and respectively each of the other N−1 selected frames. Another possibility is to form a temperature map between each pair of consecutive frames of the N frame series. In any event, the temperature maps, and ultrasound B-mode images, may both be presented as movies in real time on a display 254 that is part of the scanner 226. The temperature maps and concurrent B-mode images may be separate, e.g., side-by-side, or the temperature maps may be overlaid on the B-mode images.

In another such embodiment, a new reference frame is selected from the second file of each pair of files being compared. In particular, the new reference frame, each time, is the frame selected in the just-previous frame selection. Thus, if frame j of file L is compared with each frame of file L+1, it is because frame j, now the reference frame, was the best matching frame from file L in the previous frame selection. Thus, as in the embodiment immediately above, the first frame selection makes use of an M×M matrix, but the subsequent frame selections are each based on respective 1×M matrices. A temperature map can thus be formed between each pair of consecutive frames of the N frame series.

Alternatively, the pair-wise frame selection for the temperature maps considers, each time, all frames of both files; but it is, each time, the first file 315 of the N-file scan which is the first file of each pair of files being compared. Accordingly, there are N−1 M×M matrices. Temperature maps are formable between each pair of best matched frames, or, alternatively, between selected frames of consecutive files.

All of the above embodiments use zero-lag cross-correlation to identify cross-cycle same-phase imaging.

Another approach, however, is to search, over a maximum correlation lag, out of phase. In this approach, the correlation is not done piece-wise per frame; instead, a single region of comparison of one frame is cross-correlated over a search area in the other frame. The search area should be kept small enough that inter-image overlap still provides a sufficiently wide temperature map for ablation extent determination. The region of comparison can be two-dimensional or three-dimensional for searching correspondingly with two maximum lags or three maximum lags. The best match generally might still be at zero lag, but the contingency of bad acoustic contact, by the probe 136, at a particular cyclical phase can be accurately accommodated with a slightly out-of-phase frame.

In this approach, two regions of comparison 317, 318 can be matched if they both reside in a common search area 320. The dotted lines 322, 324 delimit image content, most of which is in one frame 326. The full image content, or a similar version, is in the other frame 328. A lagged cross-correlation (LCC) 330 experiences a maximum correlation coefficient value at a particular lag 332, in the simplified case presented in FIG. 3 of one-dimensional searching. An overlap region 334 of the two frames 326, 328, which extends from the dotted line 322 rightward to the parallel, equal-length solid line, is usable in forming a temperature map of the same spatial extent as the overlap region. Subsequent searches (i.e., to respectively determine frames 3 through N) during the multi-file scan may achieve optimality at zero lag if inter-file matching is restricted to consecutive files 218, 220 and if a reference frame is always matched to the M frames of the current file. For those times when zero lag is found to be optimal, there exists a tendency for no, or very little, further region narrowing being introduced on account of overlap. This same tendency exists in the case of a single reference frame (e.g., in the very first file) being used for all frame matching in the subsequent searches that respectively determine frames 3 through N.

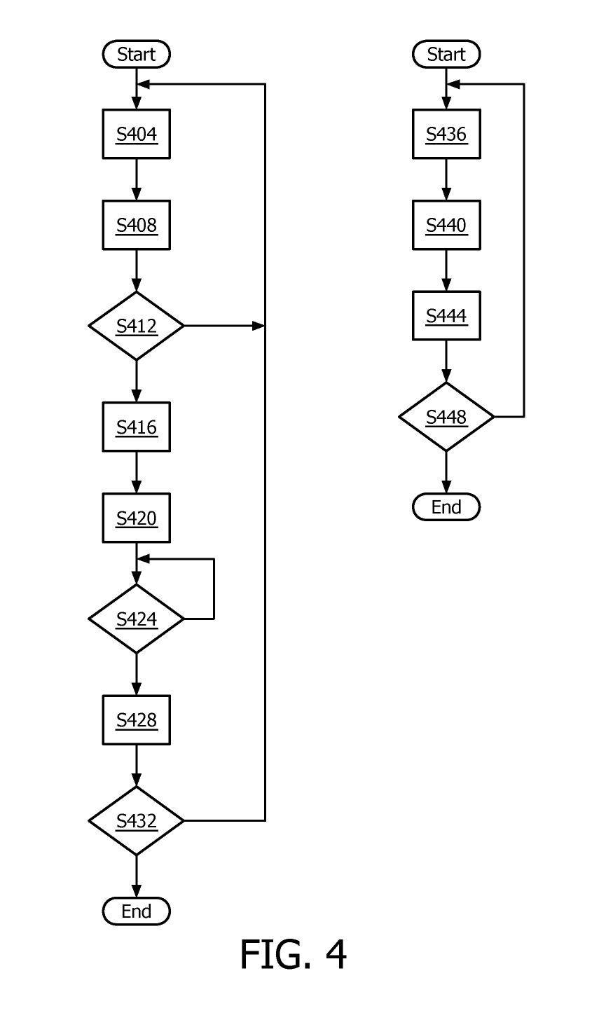

Operationally and with reference to FIG. 4 as an example, movement with a large cyclical component is detected via the respiratory belt 128 (step S404). The respiration recording device 132 records the movement (step S408). These two steps are repeated until the movement detection processor 104 detects a local peak 222 (step S412). When the peak 222 is detected (step S412), the movement detection processor 104 issues the trigger 224 a fixed time after the detection (step S416). The image acquisition circuitry 108 emits ultrasound to begin image acquisition a fixed time after the trigger 224 (step S420). When the current period 212 of acquisition expires (step S424), acquisition is interrupted (step S428). If the procedure is to continue (step S432), return is made to the movement detection step S404.

In a concurrent routine, the image matching processor 112 executes a frame selection algorithm to find a matched frame 210 in the current file 220 (step S436). The image monitoring processor 116 executes a temperature estimation algorithm using, as input, the found frame and a previous frame (step S440). The image monitoring processor 116 operates the display 264 to present the temperature map formed based on output of the temperature estimation algorithm and optionally to present a corresponding stored B-mode image (step S444). If the procedure is to continue (step S448), return is made to matched-frame finding step S436.

The mode for applying energy for heating has been described above as radiofrequency ablation (RFA). However, it is within the intended scope of what is proposed herein that ablation may be done otherwise, as by focusing a laser beam. In such a case, the chemical composition of body tissue in the path of the beam is determinable via the temperature maps. Ablating biological tissue changes its chemical composition, although not necessarily its echogenicity. However, light absorption is changed. The extent of ablation is determinable at least in the path of the laser beam. Savery relates to using monochromatic light and a temperature map in material composition analysis. The parts of Savery not incorporated by reference herein above are hereby incorporated by reference. An indicator of the extent is likewise displayable in real time, on the display 254, either on the temperature map or a B-mode image. As mentioned herein above, the map and concurrent image may be presented as separate, such as side by side, or the map may be overlaid on the B-mode image.

Although methodology of the present invention can advantageously be applied in providing medical treatment to a human or animal subject, the scope of the present invention is not so limited. More broadly, techniques disclosed herein are directed to phase-specific-view stabilization of an image depicting an object moving essential in a cyclical manner.

While the invention has been illustrated and described in detail in the drawings and foregoing description, such illustration and description are to be considered illustrative or exemplary and not restrictive; the invention is not limited to the disclosed embodiments.

For example, in the RF acquisition is step S420, the raw signal after beamforming can be saved for signal processing later. As another example, the imaging probe 136 can be a linear, convex (or “curvilinear”), phased array, matrix, transthoracic (TTE), or transesophageal (TEE) probe. In yet another example, the communicative connection between the sensor 124 and the respiratory belt 124 may be such that the apparatus 100 is configured with the sensor, positioned remotely from the belt, optically monitoring belt movement.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. Any reference signs in the claims should not be construed as limiting the scope.

A computer program can be stored momentarily, temporarily or for a longer period of time on a suitable computer-readable medium, such as an optical storage medium or a solid-state medium. Such a medium is non-transitory only in the sense of not being a transitory, propagating signal, but includes other forms of computer-readable media such as register memory, processor cache and RAM.

A single processor or other unit may fulfill the functions of several items recited in the claims. The mere fact that certain measures are recited in mutually different dependent claims does not indicate that a combination of these measures cannot be used to advantage.

1-24. (canceled)

25. A system for gating image motion, system comprising a processor; and
storage coupled to said processing unit for storing instructions that when executed by the processor cause the processor to:
detect a specific movement of an object during a movement cycle;
commence imaging of the object at a first fixed time after the specific movement is detected;
interrupt said imaging at a second fixed time after commencement of imaging and during the movement cycle, thereby imaging a pre-defined phase of the movement cycle between the first and second fixed times;
repeat the detecting, commencing, and interrupting steps during at least one other movement cycle; and
compare a plurality of images acquired from the two or more movement cycles to match one or more images of the pre-defined phase.

26. The system of claim 25, wherein the processor is further configured to generate a temperature map from the matched images.

27. The system of claim 25, wherein the movement cycles correspond to respiratory cycles.

28. The system of claim 26, wherein the specific movement comprises a peak movement within a respiratory cycle.

29. The system of claim 25, wherein the imaging comprises imaging with an ultrasound transducer.

30. The system of claim 25, wherein the images acquired from each movement cycle comprise the same pre-defined phase.

31. The system of claim 25, wherein comparing step comprises matching images based on a static region in the plurality of images.

32. The system of claim 25, wherein the object comprises tissue.

33. The system of claim 32, wherein at least a portion of the tissue is being ablated.

34. A computer-readable medium embodying a program having instruction executable by a processor for performing a method comprising the steps of:
detecting a specific movement of an object during a movement cycle;
commencing imaging of said object at a first fixed time after the specific movement is detected;
interrupting said imaging after a second fixed time after commencement of imaging and during the movement cycle, thereby imaging a pre-defined phase of the movement cycle between the first and second fixed times;
repeating the detecting, commencing, and interrupting steps during at least one other movement cycle; and
comparing a plurality of images acquired from the two or more movement cycles to match images based on a static region in the plurality of images.

35. The computer-readable medium of claim 34, wherein the method further comprises the step of generating a temperature map from the matched images.

36. The computer-readable medium of claim 34, wherein the movement cycles correspond to respiratory cycles.

37. The computer-readable medium of claim 34, wherein the specific movement comprises a peak movement within a respiratory cycle.

38. The computer-readable medium of claim 34, wherein the imaging comprises imaging with an ultrasound transducer.

39. The computer-readable medium of claim 25, wherein the images acquired from each movement cycle comprise the same pre-defined phase.

40. The computer-readable medium of claim 25, wherein comparing step comprises matching images based on a static region in the plurality of images.

41. The computer-readable medium of claim 25, wherein the object comprises tissue.

42. The computer-readable medium of claim 41, wherein at least a portion of the tissue is being ablated.
